(19) United States
(12) Patent Application Publication
(10) Pub. No.: US 2013/0281742 A1
(43) Pub. Date: Oct. 24, 2013

(54) METHOD AND SYNTHESIS OF INITIATORS FOR TELECHELIC POLYISOBUTYLENES

(71) Applicants: Joseph P. Kennedy, Akron, OH (US); Gabor Erdodi, Stow, OH (US)

(72) Inventors: Joseph P. Kennedy, Akron, OH (US); Gabor Erdodi, Stow, OH (US)

(73) Assignee: The University of Akron, Akron, OH (US)

(21) Appl. No.: 13/977,941

(22) PCT Filed: Dec. 30, 2011
(86) PCT No.: PCT/US11/68104

§ 371 Date: Jul. 2, 2013

Related U.S. Application Data
(60) Provisional application No. 61/429,334, filed on Jan. 3, 2011.

(51) Int. Cl. C07C 41/01

(52) U.S. Cl. CPC C07C 41/01
USPC 568/662

(57) ABSTRACT

A new methodology for the synthesis of a novel difunctional- and a known trifunctional initiator, i.e., 1,3-di(2-methoxy-2-propyl)-5-isopropyl benzene and 1,3,5-tri(2-methoxy-2-propyl)benzene, respectively, for the preparation of di- and tri-telechelic polyisobutylenes. The synthesis proceeds in three steps: 1) catalytic peroxidation of 1,3,5-triisopropylbenzene, 2) reduction of the peroxides to the corresponding alcohols, and 3) methylation of the alcohols. By controlling the conversion of the key peroxidation step the relative ratio of di- and tri-functional intermediates can be controlled. By the use of the 1,3-di(2-methoxy-2-propyl)-5-isopropyl-benzene, well-defined di-methoxy telechelic polyisobutylenes can be synthesized. Although the overall combined yield of the two initiators was only 14-20%, because of the low cost of the starting material, reagents used, and simple manipulations these compounds represent the most cost effective initiators to-date for the preparation of telechelic polyisobutylenes.

### CROSS-REFERENCE TO RELATED APPLICATIONS

This application is a 371 National Phase application of PCT application No. PCT/US2011/68104, filed Dec. 30, 2011, which claims the benefit of U.S. Provisional Patent Application No. 61/429,334, filed Jan. 3, 2011, now abandoned.

### TECHNICAL FIELD

The present invention relates to a method for synthesizing a new di-functional initiator or a known tri-functional initiator. More particularly, the present inventions relates to a method for the synthesis of 1,3(2-methoxy-2-propyl)-5-isopropyl-benzene and 1,3,5-tri-(2-methoxy-2-propyl)benzene, which initiators are then used for living carbocationic polymerizations, such as the preparation of di- and tri-telechelic polyisobutylenes. The resultant, new di-functional initiator, 1,3(2-methoxy-2-propyl)-5-isopropyl-benzene, is also claimed.

### BACKGROUND FOR THE INVENTION

The invention of living carbocationic polymerizations, specifically that of isobutylene, was a milestone in synthetic polymer science because, in addition to a synthetic breakthrough, it lead to the development of several commercially significant products. One of these products is telechelic polyisobutylene (F—PIB—F, where F=functional group, PIB=polyisobutylene), the enabling intermediate of poly(styrene-b-isobutylene-b-styrene) (SIBS), the drug eluting coating on Boston Scientific's Taxus® coronary stent implanted in and enhancing the quality of life of millions of people!

A cost analysis of the product revealed that up to 90% of the cost of the commercially significant low molecular weight (defined herein as Mn˜3000 g/mol or less) telechelic polyisobutylene is due to the “blocked” initiator 1,3(2-methoxy-2-propyl)-5-tert butylbenzene (hereinafter “tBuDiCumMeO”) employed for its synthesis. The reasons for using this specific structure have been presented is well known, but to date, no other structures have been commercially available and/or commercially successful for the specific use as a polymerization initiator for the specific living carbocationic polymerizations desired.

Heretofore, however, it has not been shown that other polymerization initiators could be produced that are far less expensive and just as efficient as the initiator, tBuDiCumMeO. By providing such a polymerization initiator that is far less expensive to synthesize, but just as efficient, such as, for example, 1,3-di(2-methoxy-2-propyl)-5-isopropyl benzene (also noted sometimes hereinafter as “iPrDiCum MeO”), it is believed that alternative initiators could find commercial success in the growing industry of initiators for carbocationic polymerizations.

Thus, a need exists for a new simple low cost synthesis of novel polymerization initiators. One such functional initiator, iPrDiCum MeO, together with the trifunctional initiator 1,3,5(2-methoxy-2-propyl)benzene| (hereinafter sometimes referred to as “triCumMeO”) can be used for the preparation of ditelechelic and tritelechelic polyisobutylenes, respectively. That is, the need exists for a difunctional or a trifunctional initiator suitable for the living polymerization of isobutylene to well-defined telechelic polyisobutylenes.

### SUMMARY OF THE INVENTION

Any one or more of the foregoing aspects of the present invention, together with the advantages thereof over known art relating to polymerization initiators and the methods of synthesis of the same, which will become apparent from the specification that follows, may be accomplished by the invention as hereinafter described and claimed.

The present invention provides a method for the synthesis of at least one of 1,3-di(2-methoxy-2-propyl)-5-isopropyl benzene and 1,3,5-tri(2-methoxy-2-propyl)benzene. Each composition is synthesized to be of high purity. By the term “high purity,” it is meant that, other than the expected resonances for each material component, the NMR spectrum for the composition has no other discernable resonances higher than 0.05 normalized intensity. For compositions noted to have “very high purity,” this term is defined as, other than the expected resonances for each material component, the NMR spectrum for the composition has no other discernable resonances higher than 0.01 normalized intensity. Alternatively, where other methods of determining purity are used, the compositions in one or more embodiments of the present invention are to be 95% pure. In other embodiments, the compositions are to be 99% pure.

The present further provides a method for synthesizing 1,3-di(2-methoxy-2-propyl)-5-isopropyl benzene and/or 1,3,5-tri(2-methoxy-2-propyl)benzene comprising a number of steps, including peroxidizing 1,3,5-triisopropylbenzene by gaseous oxygen in the presence of a catalyst in a basic solution to obtain a mixture of peroxide intermediates having one, two or three carboxyl groups; reducing the mixture of peroxide intermediates and unreacted 1,3,5-triisopropyl benzene to provide a mixture of hydroxyl derivatives having one, two or three hydroxyl groups, unreacted peroxide intermediates and unreacted 1,3,5-triisopropylbenzene, wherein the hydroxyl derivative having two hydroxyl groups is 1,3,-di(2-hydroxyl-2-propyl)-5-isopropylbenzene, and wherein the hydroxyl derivative having three hydroxyl groups is 1,3,5-tri(2-hydroxyl-2-propyl)benzene; separating by solvent precipitation the 1,3-di(2-hydroxyl-2-propyl)-5-isopropyl benzene and the 1,3,5-tri(2-hydroxyl-2-propyl)benzene formed in the step of reducing from any other derivatives, intermediates and unreacted materials in the reduced mixture to form a mixture of 1,3-di(2-hydroxyl-2-propyl)-5-isopropyl benzene and 1,3,5-tri(2-hydroxyl-2-propyl)benzene; methylating a mixture of 1,3-di(2-hydroxyl-2-propyl)-5-isopropylbenzene and 1,3,5-tri(2-hydroxyl-2-propyl)benzene mixture to form a mixture of 1,3-di(2-methoxy-2-propyl)-5-isopropyl benzene and 1,3,5-tri(2-methoxy-2-propyl)benzene; separating the 1,3-di(2-methoxy-2-propyl)-5-isopropylbenzene from the 1,3,5-tri(2-methoxy-2-propyl)benzene; and purifying the separated 1,3-di(2-methoxy-2-propyl)-5-isopropyl benzene and the 1,3,5-tri(2-methoxy-2-propyl)benzene by crystallization from an organic solvent. In one or more embodiments, the catalyst in the basic solution may be a Cobalt(II) salt and a tertiary amine in water. In that embodiment, or in other embodiments, the mixture may be reduced using sodium sulfite. In one or more embodiments, including any of the preceding embodiments, the step of separating by solvent precipitation may use hexane as the solvent. In one or more embodiments, including any of the preceding embodiments, the step of separating the 1,3-di(2-methoxy-2-propyl)-5-isopropylbenzene from the 1,3,5-tri(2-methoxy-2-propyl)benzene may be carried out by vacuum distillation at different boiling points for each material. In one or more embodiments, including any of the preceding embodiments, the step of purifying may be carried out using hexane as the organic solvent.

A further aspect of the present invention provides a novel composition of matter, namely, 1,3-di(2-methoxy-2-propyl)-5-isopropylbenzene, for the preparation of telechelic polyisobutylenes by living carbocationic polymerization. This new structure preserves the initiating cumyl groups in the 3 and 5 positions on the aromatic ring of the commonly used initiator tBuDiCumMeO, and contains an isopropyl blocking substituent in the 1 position to prevent intramolecular alkylation by steric inhibition.

Beneficially, it will be appreciated that from the synthesis of these compositions, namely, highly pure 1,3-di(2-methoxy-2-propyl)-5-isopropyl benzene and/or highly pure 1,3,5-tri(2-methoxy-2-propyl)benzene, low cost polymerization initiators may be produced that are highly desirable and efficient in carrying out living carbocationic polymerizations. That is, polymerization initiators suitable for use in the preparation of living carbocationic polymers, such as, for example, ditelechelic and tritelechelic polyisobutylenes, can be synthesized.

### BRIEF DESCRIPTION OF THE DRAWINGS

FIG. 1 is a 1H NMR spectrum of a mixture of unreacted 1,3,5-triisopropylbenzene and mono-, di-, and tri-functional 2-peroxy-2-propylbenzenes;

FIG. 2 is a chromatogram of the peroxidized mixture containing unreacted 1,3,5-triisopropylbenzene (1), mono-functional (2), di-functional (3), and tri-functional (4) 2-peroxy-2-propylbenzenes, with the nature of the impurity at about 5.5 min. unknown;

FIG. 3 is a C13 NMR spectrum of the mixture of unreacted 1,3,5-triisopropylbenzene, mono-(2-hydroxyl-2-propyl)-3,5-diisopropylbenzene, 1,3,-di(2-hydroxyl-2-propyl)-5-isopropylbenzene, and 1,3,5-tri(2-hydroxyl-2-propyl)benzene, wherein the inset shows the spectrum of the original mixture;

FIG. 4 is a 1H NMR spectrum of 1,3,-di(2-methoxyl-2-propyl)-5-isopropylbenzene;

FIG. 5 is a 1H NMR spectrum of 1,3,5-tri(2-methoxyl-2-propyl)benzene; and

FIG. 6 is a GPC trace of a representative telechelic PIB synthesized by the use of 1,3,-di(2-methoxyl-2-propyl)-5-isopropylbenzene.

### BRIEF DESCRIPTION OF AN ILLUSTRATIVE EMBODIMENT

The present invention provides for a new, low cost method for producing desirable and efficient polymerization initiators for living carbocationic polymerizations. In doing so, a novel composition has been developed. The new di-functional composition, as well as the known tri-functional composition produced are desirably of high purity and suitable for use as initiators for polymerizations such as those that produce telechelic polyisobutylenes, wherein the di-functional initiator will be used to produce ditelechelic polyisobutylenes and the tri-functional initiator will be used to produce tritelechelic polyisobutylenes.

The initiators produced are 1,3-di(2-methoxy-2-propyl)-5-isopropyl benzene and 1,3,5-tri(2-methoxy-2-propyl)benzene. It will be appreciated that the former composition is a novel composition and may also have the name 1-isopropyl-3,5-dicumylmethyl ether. Both compositions can be made with high purity as defined hereinabove. In one embodiment, the compositions are made to be 95% pure. In another embodiment, the compositions are made to be 99% pure. It is believed that even the known composition, 1,3,5-tri(2-methoxy-2-propyl)benzene, has heretofore not been made this pure.

In order to understand the synthesis of the compositions, it will be appreciated that Scheme 1 hereinbelow provides one detailed embodiment of a suitable reaction scheme for the present invention. It will be appreciated that Scheme 1 outlines the synthesis of 1,3-di(2-methoxy-2-propyl)-5-isopropyl benzene (referred to herein and the Scheme 1 as “[OMe]2”) and 1,3,5-tri(2-methoxy-2-propyl)benzene (referred to herein and in Scheme 1 as “[OMe]3”), as described herein, and should be referred to throughout this discussion of the synthesis of these compositions.

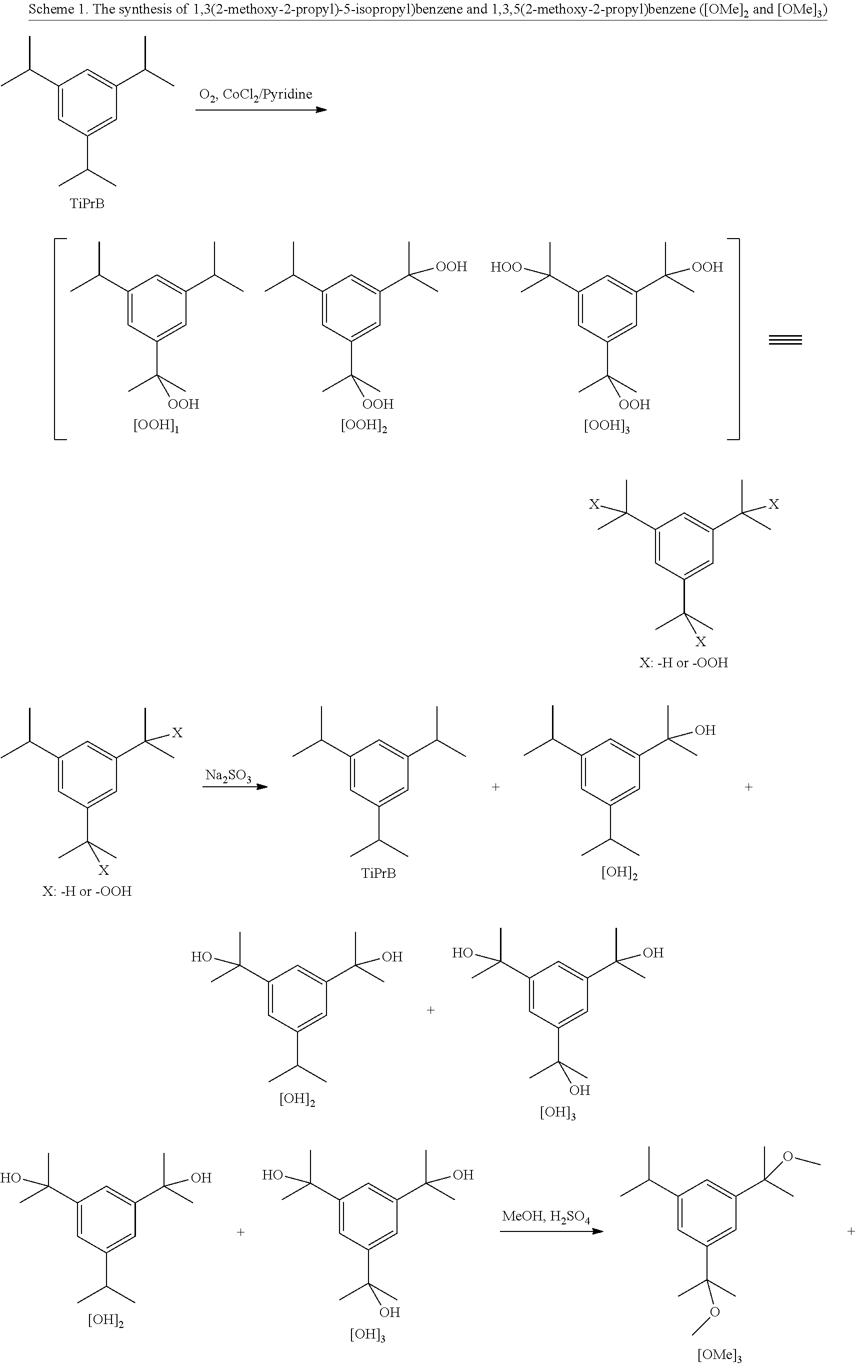

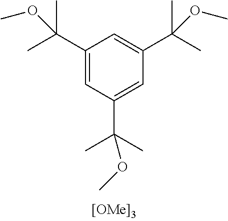

The method for synthesizing 1,3-di(2-methoxy-2-propyl)-5-isopropyl benzene and/or 1,3,5-tri(2-methoxy-2-propyl)benzene comprises, as a first step, peroxidizing 1,3,5-triisopropylbenzene (referred to herein and in Scheme 1 as “TiPrB”) by gaseous oxygen in the presence of a catalyst in a basic solution. In one embodiment, the catalyst in the basic solution may be a Cobalt(II) salt and a tertiary amine in water. In other embodiments, the catalyst in the basic solution may be cobalt chloride in pyridine in water at 90° C. Amounts of catalyst and quantities of basic solution are well known to the skilled artisan in conducting peroxidation reactions and can be done without undue experimentation.

In one embodiment, the de facto catalysts are cobalt (II) oxide/hydroxide complexes that form in situ from CoCl₂ under basic conditions during O₂ bubbling. Although the catalyst particles are insoluble in water, they remain dispersed in the aqueous phase. The reaction may be carried out in the presence of pyridine as co-catalyst and improves the adsorption of the reactants on the catalyst particles. The pH of the solution strongly influences both the yield and reaction rate. At low pH, the reaction is rapid but the yield of peroxides decreases because other oxidized byproducts are also formed. It was found that pH=10 is optimal for the synthesis. The medium becomes slowly acidic due to the formation of peroxides. To avoid undesirable pH changes, the pH was adjusted periodically by KOH and a small amount of Na₂HPO₄ buffer was added.

As a result of the peroxidation of TiPrB, a mixture of peroxide intermediates having one, two or three carboxyl groups are produced, together with unreacted TiPrB. These peroxide intermediates are mono-functional, di-functional, and tri-functional 2-peroxy-2-propylbenzenes, which are referred to herein and in Scheme 1 as [OOH]1, [OOH]2, AND [OOH]3, respectively.

This mixture of peroxide intermediates and unreacted 1,3,5-triisopropyl benzene (schematically, TiPrB+[OOH]1+[OOH]2+[OOH]3) is then reduced, by way of one or more known processes for reducing organic mixtures. In one embodiment, the mixture may be reduced using sodium sulfite. The reduction may take place in water at room temperature. In another or the same embodiment, to avoid autoaccerlerating self-oxidation of the peroxides, the mixture may be added dropwise to a slightly acidic or neutral sodium sulfite solution so that no reaction occurs. Again, amounts of reducing agent needed for carrying out this reaction will be known to those skilled in the art and can be carried out without undue experimentation.

The resultant reduction provides a mixture of hydroxyl derivatives having one, two or three hydroxyl groups, possibly some unreacted peroxide intermediates and unreacted 1,3,5-triisopropylbenzene. This reduction step is generally shown in the second line of Scheme 1 above, wherein the peroxide intermediates and unreacted TiPrB mixtures is reduced to a mixture defined as unreacted TiPrB, a hydroxyl derivative having one hydroxyl group, (referred to in Scheme 1 and hereinafter as “[OH]1”), a hydroxyl derivative having two hydroxyl groups, namely, 1,3,-di(2-hydroxyl-2-propyl)-5-isopropylbenzene, (referred to in Scheme 1 and hereinafter as “[OH]2”), and a hydroxyl derivative having three hydroxyl groups, namely, 1,3,5-tri(2-hydroxyl-2-propyl)benzene (referred to in Scheme 1 and hereinafter as “[OH]3”). It will be understood that it is possible that there may be some unreacted [OOH]1, [OOH]2 and [OOH]3 in this mixture as well, although subsequent NMR spectra shows most of these peroxide intermediates are reduced and converted to their corresponding hydroxyl derivatives.

The method continues by separating the 1,3-di(2-hydroxyl-2-propyl)-5-isopropyl benzene ([OH]2) and the 1,3,5-tri(2-hydroxyl-2-propyl)benzene ([OH]3) formed in the step of reducing from any other derivatives, intermediates and unreacted materials in the reduced mixture. In one embodiment, this is done by solvent precipitation. In one embodiment, the solvent is an organic solvent. In another embodiment, the solvent is hexane. TiPrB and [OH]1 are hexane soluble liquids, while [OH]2 and [OH]3 are insoluble in hexane and are crystalline solids. Thus, separation of these mixtures is ready completed by precipitation of into hexanes. The by-products of the peroxidation and reduction reactions remain generally in the hexane, while the impurities in the [OH]2 and [OH]3 mixture can be removed with ether.

The mixture of 1,3-di(2-hydroxyl-2-propyl)-5-isopropylbenzene and 1,3,5-tri(2-hydroxyl-2-propyl)benzene then undergo methylation. Methylating the mixture of [OH]2 and [OH]3 forms a mixture of 1,3-di(2-methoxy-2-propyl)-5-isopropyl benzene ([OMe]2) and 1,3,5-tri(2-methoxy-2-propyl)benzene ([OMe]3), as shown in the last line in Scheme 1. Any known method of methylation may be used for the present invention. In one embodiment, methylation is carried out in the presence of an H₂SO₄ catalyst in methanol.

After methylation, the resultant compositions desired may be separated. That is, the 1,3-di(2-methoxy-2-propyl)-5-isopropylbenzene may be separated from the 1,3,5-tri(2-methoxy-2-propyl)benzene by vacuum distillation, as the two compositions have different boiling points. The boiling point of [OMe]2 is 75° C., while the boiling point of [OMe]3 is 105° C. at 0.5 mbar. Thus, vacuum distillation provides a good and efficient method for separation.

Finally, the separated 1,3-di(2-methoxy-2-propyl)-5-isopropyl benzene and the 1,3,5-tri(2-methoxy-2-propyl)benzene may be purified. During methylation, small amounts of olefin side products are formed by loss of water, which cannot be separated by vacuum distillation. Thus, the final products are purified by crystallization from an organic solvent. In one embodiment, the step of purifying may be carried out using hexane as the organic solvent.

It will be appreciated that the resultant compositions can be used as suitable polymerization initiators for the preparation of telechelic polyisobutylenes. Reaction of the intiator [OMe]2 and, separately, [OMe]3 with isobutylene can be carried out as well known in the art for such living carbocationic polymerizations. The preparation of allyl-telechelic polyisobutylenes (allyl-PIB-allyl) and Ø(PIB-allyl)₃ are well known and have been described throughout the literature.

In order to demonstrate practice of the invention, the method described above and as set forth in Scheme 1 was used to prepare the compositions 1,3-di(2-methoxy-2-propyl)-5-isopropylbenzene and 1,3,5-tri(2-methoxy-2-propyl)benzene. In preparation for conducting the method, 1,3,5-triisopropylbenzene (TiPrB) was obtained from TCI America, CoCl₂×6H₂O, pyridine, Na₂HP04×7H₂O, KOH, N,N,N′,N′-tetramethylethane-1,2-diamine, and Na₂SO₃ were obtained from Aldrich. Tetrahydrofuran, diethyl ether, hexanes, methanol, CH₂Cl₂, NaHCO₃, MgSO₄ and sulfuric acid were obtained from Fischer. Isobutylene and oxygen were obtained from Praxair.

All 1H and C13 NMR spectra were obtained by a Varian Mercury 300 MHz NMR spectrometer in deuterated chloroform solutions. GPC eluograms were obtained with a Waters GPC instrument equipped with a series of six Waters Styragel columns (HR 0.5, HR 1, HR 3, HR 4, HR 5, and HR 6) and a refractive-index detector (Optilab, Wyatt Technology). Samples were dissolved in THF, and the flow rate was 1 mL THF/min. Molecular weights were calculated by the use of polystyrene standards. Gas chromatograms were obtained by a Shimadzu instrument equipped with an Equity-1 fused silica capillary column, a TCD detector, and a CR501 recorder using He as carrier gas.

As presented in Scheme 1 and generally explained above, one representative embodiment for carrying out the synthesis of 1,3-di(2-methoxy-2-propyl)-5-isopropylbenzene and 1,3,5-tri(2-methoxy-2-propyl)benzene is described and carried out in the following detailed steps.

### The Synthesis of the TiPrB+[OOH]1+[OOH]2+[OOH]3 Mixture

In a 3 neck 1 L flask equipped with a condenser, a mechanical stirrer and a gas inlet were placed. CoCl₂×6H₂O (15 g, 0.063 mol) and pyridine (30 g, 0.38 mol) were dissolved in 200 mL water. In a beaker, KOH (7 g, 0.125 mol) and Na₂HPO₄×7H₂O (20 g, 0.075 mol) were dissolved in 200 mL water, and the solution was added to the contents of the flask together with 1,3,5-triisopropylbenzene (100 g, 0.49 mol) during stirring. Oxygen was bubbled into the solution at ˜30 L/h and the system was heated to 90-95° C. during strong stirring. During the peroxidation, the pH was monitored and maintained at pH=10 by the periodic addition of a KOH solution (typically 3 g, 0.05 mol KOH in 10 mL water was added over the course of 5 h; during this period the conversion as determined by ¹H NMR reached 65%). The reaction was stopped by cooling the system when the desired conversion was reached. The viscous TiPrB+[OOH]1+[OOH]2+[OOH]3 slurry was separated from the aqueous phase and extracted with 2-300 mL ether. The ether-soluble fraction ([OOH]2+[OOH]3) was used in the next step without further purification.

### The Synthesis of the [OH]₂+[OH]₃ Mixture

The ether-soluble fraction was added dropwise to a charge of 190 g Na₂SO₃ in 500 mL water placed in a 3 neck 1 L flask equipped with a mechanical stirrer and a thermometer. The temperature of the solution was maintained at −20° C. and the pH was kept at pH=6-7. The solution of 1,3,5-(2-peroxy-2-propyl)benzene mixture, i.e., the peroxide intermediates mixture, obtained in the previous step was added to the Na₂SO₃ in 30 minutes at 10-20° C. and at pH=6-7 during strong stirring. The solution was stirred for an additional 30 minutes at room temperature, the organic phase was separated, the aqueous phase was extracted with additional ether. The organic phases were combined, washed with NaHCO₃ and dried over MgSO₄. The ether was evaporated, the viscous liquid obtained (105 g) was mixed with 400 mL hexanes, and stirred for 30 min at room temperature. The hexane insoluble [OH]2 and [OH]3 were filtered off and purified by ether extraction (200 mL). The filtrate, a mixture of [OH]2 and [OH]3, was dried in vacuum for 24 hours and yielded 47 g, for a conversion of approximately 40%.

### The Synthesis of the Final Products: [OMe]₂ and [OMe]₃

The [OH]2+[OH]3 mixture (see above) was dissolved in 500 mL methanol, and 0.1 g H₂SO₄ was added. The solution was kept at 60° C. for 16 hours. 10 mL water was added, and the methanol was extracted with hexanes. The hexanes solution of [OMe]2+[OMe]3 mixture was washed with water and dried over MgSO₄. The hexanes solvent was evaporated and the mixture was distilled in vacuum at 0.5 mbar. The [OMe]2 fraction was collected at 75° C., and the [OMe]3 fraction was collected at 105° C. Both fractions were purified by crystallization in hexanes. [OMe]2 yielded 22 g of white crystals (m.p.: 28° C.) of 99% purity (by GC), and [OMe]3 yielded 5 g of white crystals of 98% purity (by GC).

As a result of this method, tests were conducted to confirm the compositions obtained. One desirable aspect of the present invention was the synthesis of a new inexpensive “blocked” initiator, 1,3-di(2-methoxy-2-propyl)-5-isopropyl benzene, (abbreviated [OMe]2 in Scheme 1) for the preparation of telechelic polyisobutylenes by living carbocationic polymerization. This new structure preserves the initiating cumyl groups in the 3 and 5 positions on the aromatic ring of the commonly used initiator tBuDiCumMeO, and contains an isopropyl blocking substituent in the 1 position to prevent intramolecular alkylation by steric inhibition. Another desirable aspect of the present invention was the synthesis of the known trifunctional initiator [OMe]3 for the preparations of tritelechelic polyisobutylenes.

### 1. Peroxidation

The synthesis started with the catalytic peroxidation of TiPrB by gaseous oxygen in the presence of CoCl₂, pyridine in water at 90° C. The de facto catalysts are cobalt (II) oxide/hydroxide complexes that form in situ from CoCl₂ under basic conditions during O₂ bubbling. Although the catalyst particles are insoluble in water, they remained dispersed in the aqueous phase. The reaction was carried out in the presence of pyridine as co-catalyst and improved the adsorption of the reactants on the catalyst particles. The pH of the solution strongly influenced both the yield and reaction rate. At low pH, the reaction was rapid, but the yield of peroxides decreased because other oxidized byproducts were also formed. It was found that a pH=10 was optimal for this synthesis. The medium slowly became acidic due to the formation of peroxides. To avoid undesirable pH changes, the pH was adjusted periodically by KOH and a small amount of Na₂HPO₄ buffer was added.

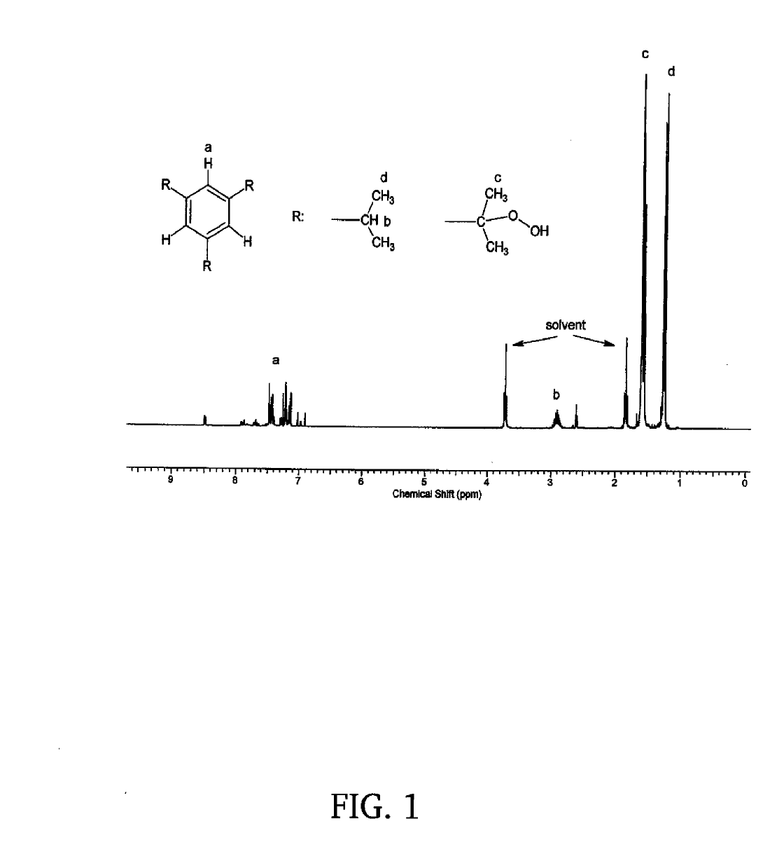

FIG. 1 shows the ¹H NMR spectrum of the mixture containing unreacted starting material together with mono- di- and trifunctional 2-peroxy-2-propylbenzenes (TiPrB+[OOH]1+[OOH]2+[OOH]3) mixture; see first line of Scheme 1). obtained after 5 hours at 65% conversion. Conversion was calculated from the ratio of the resonance integrals at 1.3 and 1.6 ppm, associated with the methyl groups of the original isopropyl and the oxidized 2-peroxy-2-propyl groups, respectively.

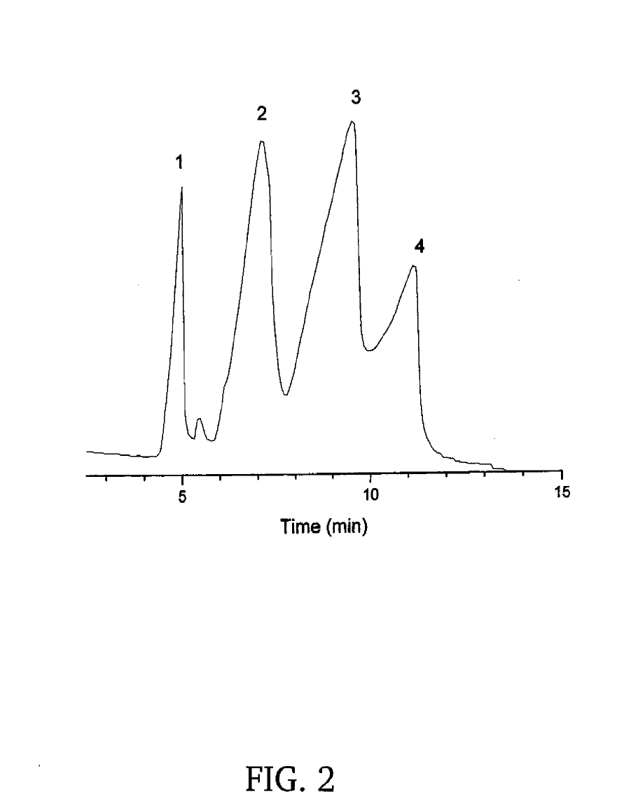

FIG. 2 shows the gas chromatogram of the peroxydized mixture. The four peaks correspond to the unreacted TiPrB and the three peroxydized products fitted with one ([OOH]1), two ([OOH]2), and three ([OOH]3) 2-peroxy-2-propyl groups. The nature of the impurity at ˜5.5 min is unknown.

2. Reduction Followed by Separation of [OH]2 and [OH]3 by Precipitation into Hexanes

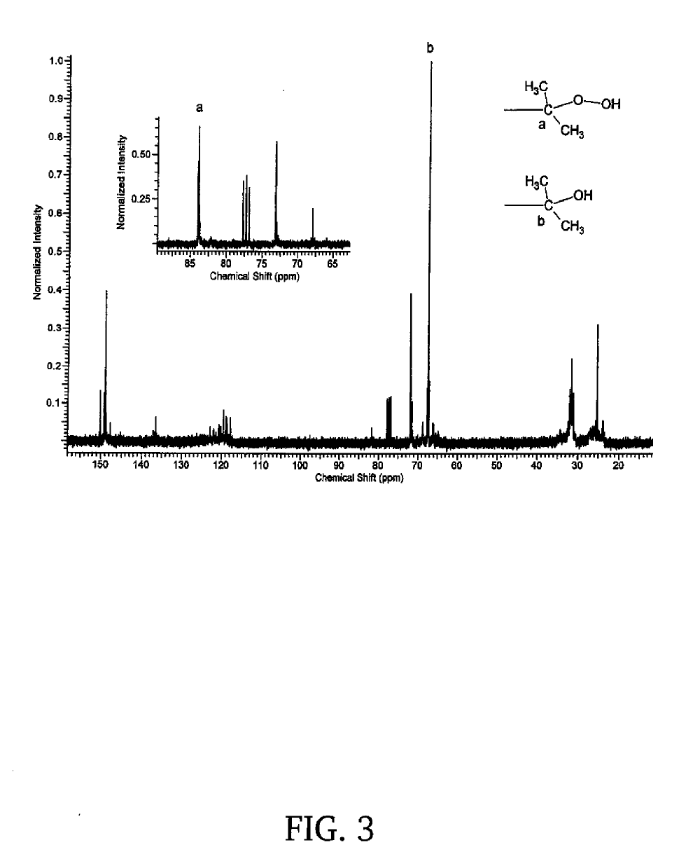

The second step of the synthesis was the reduction of the mixture of the peroxides by NaSO₃ to the corresponding hydroxyl derivatives in water at room temperature. To avoid the autoaccelerating self-oxidation of the peroxides, the mixture was added dropwise to a slightly acidic or neutral Na₂SO₃ solution (at pH>7 so that no reaction occurred. TiPrB and [OH]1 are hexanes-soluble liquids while [OH]2 and [OH]3 are hexanes insoluble crystalline solids; thus the separation of these mixtures was readily completed by precipitation into hexanes. The byproducts of peroxidation/reduction remained mainly in the hexanes and the impurities in the [OH]2+[OH]3 mixture can be removed by extraction with ether. FIG. 3 shows the C13 NMR spectrum of the mixture before and after reduction. The resonance at 84 ppm associated with the carbon adjacent to the peroxide group disappears after reduction, which indicates that the reduction was complete.

### 3. Methylation

The final step was the methylation of the [OH]2+[OH]3 mixture to [OMe]2+[OMe]3 (see line 3 in Scheme 1) carried out in the presence of H₂SO₄ catalyst in methanol. After methylation, the target initiators [OMe]2 and [OMe]3 were separated by vacuum distillation. The boiling points of these compounds were sufficiently different (75 and 105° C. at 0.5 mbar, respectively) for efficient rectification.

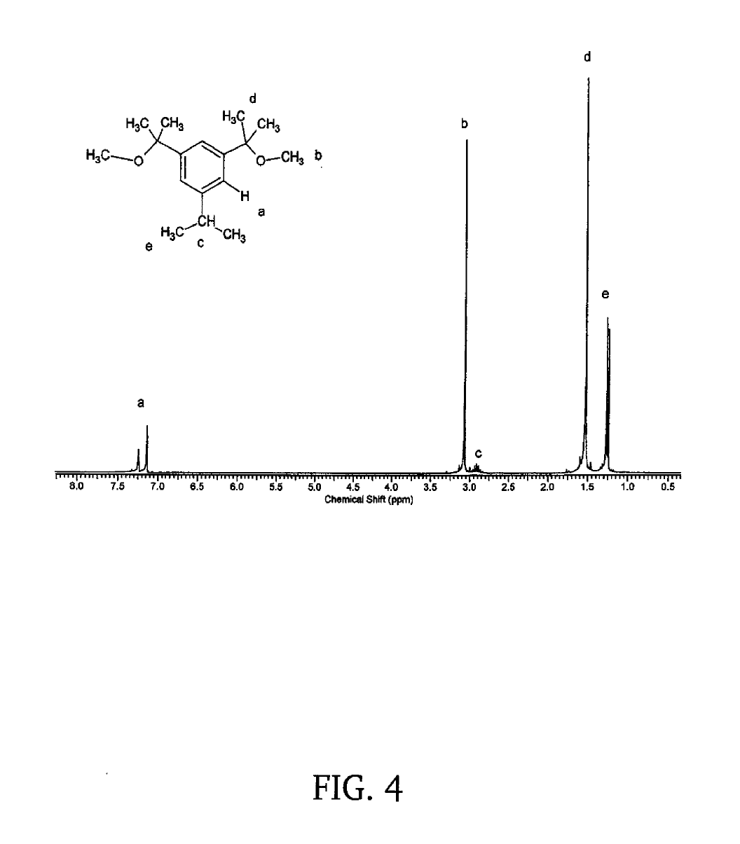

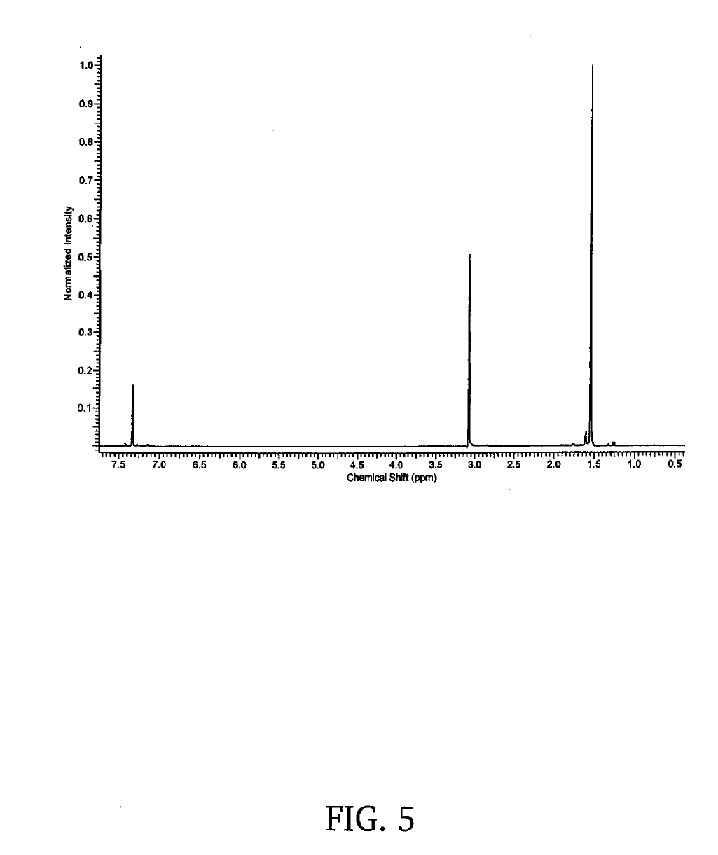

During methylation, small amounts of olefin side products were formed by loss of water, which could not be separated by distillation. Thus, the final products were obtained by crystallizations from hexanes. The purity of [OMe]2 and [OMe]3 were 99% and 98%, respectively (determined by GC). FIGS. 4 and 5 show the ¹H NMR spectra of the purified products.

### [OMe]2/[OMe]3 Ratios and Overall Yields

Due to the statistical distribution of the products of peroxidation, practically any [OOH]2/[OOH]3 ratios can be synthesized. If peroxidation is driven to approximately 100% conversion, the main product (after reduction and methylation) is [OMe]3. At 60 to 70% conversions, however, the main product is [OOH]2 and is accompanied by significant amounts of [OOH]1 together with smaller amounts of [OOH]3.

Table I provides yields of TiPrB+[OH]1 and [OH]2+[OH]3 mixtures, and [OMe]2 and [OMe]3 formed at 66 and 90% conversions.

TABLE I

| | Yields | | | | |
|---|---|---|---|---|---|
| | TiPrB + | [OH]2 + | | | [OMe]2 + [OMe]3 |
| Conv. | [OH]1 | [OH]3 | [OMe]2 | [OMe]3 | combined overall |
| % | [g] | [g] | [g] | [g] | [mol %] |
| 66 | 41 | 47 | 22 | 5 | 20 |
| 90 | 23 | 51 | 6 | 15 | 14 |

Table I shows the yields of TiPrB+[OH]1 and [OH]2+[OH]3 mixtures, and the targeted end products [OMe]2 and [OMe]3 at 66 and 90% conversions. The combined overall yield obtained at 90% conversion is 14% which is lower than that obtained at 66% conversion because of side reactions producing higher molecular weight oligomers during peroxidation. Although the combined overall yields of [OMe]2 and [OMe]3 by the new method is lower than tBuDiCumMeO and [OMe]3 obtained by a known Grignard-based method, the use of the less expensive starting material and reagents lowers the cost of synthesis of the initiator [OMe]2 to a fraction of the earlier “blocked” initiator, tBuDiCumMeO.

Peroxidation produces considerable amounts of TiPrB+[OH]1 (see Table I). While the monofunctional peroxide is a side product, the TiPrB+[OH]1 mixture can be recycled and subjected to another peroxidation, as desired. This may lead to significant increases in the yields of the more desirable [OMe]2 and [OMe]3.

### The Preparation of Telechelic Polyisobutylene by the Use of [OMe]2

The efficacy of [OMe]2 as initiator for the polymerization of isobutylene to well-defined telechelic polyisobutylene has been experimentally demonstrated. This experiment was carried with well-established polymerization conditions and polymer characterization methods.

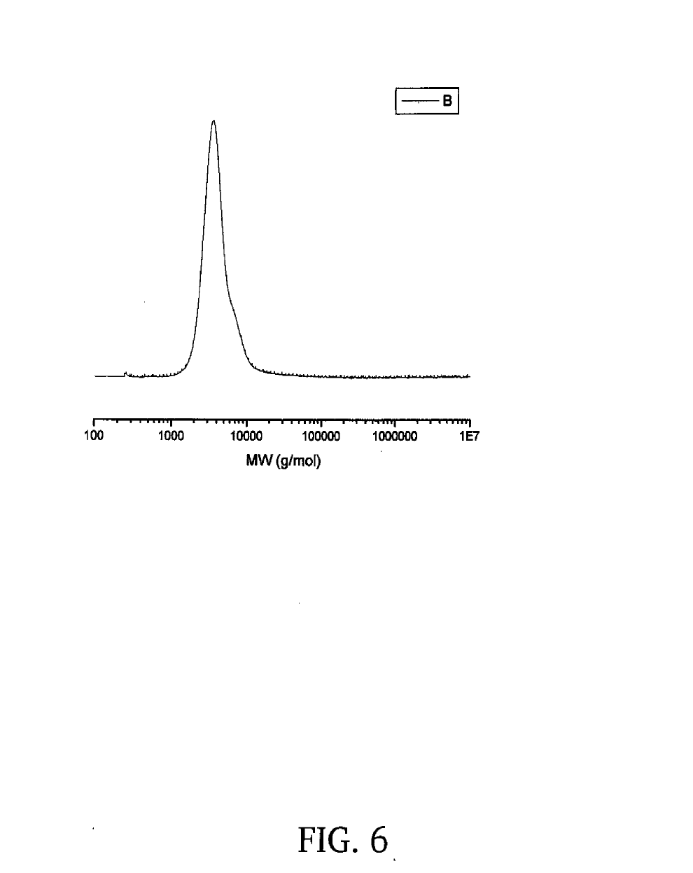

FIG. 6 shows the molecular weight distribution of a low molecular weight (2200 g/mol) allyl-telechelic polyisobutylene synthesized by using [OMe]2. As anticipated, the Mw/Mn of the polymer was 1.16 and 1H NMR spectroscopy indicated 2.0+/−0.2 terminal allyl-functionality. Accordingly, it has been demonstrated by these studies that 1,3-di(2-hydroxyl-2-propyl)-5-isopropylbenzene ([OMe]2) is a new, low cost, efficient initiator for the synthesis of telechelic polyisobutylenes. It has previously been established that [OMe]3 is such an initiator, but [OMe]3 has heretofore not been synthesize with as high a purity as when produced by the inventive method above. The Grignard reagents method does not provide such a highly pure [OMe]3.

In light of the foregoing, a novel composition of 1,3-di(2-hydroxyl-2-propyl)-5-isopropylbenzene ([OMe]2) and a known composition of 1,3,5-tri-(2-methoxy-2-propyl)benzene ([OMe]3) have been synthesized for use as “blocked” initiators for the preparation of telechelic polyisobutylenes by living carbocationic polymerization. The method of synthesis is unique to these compositions and, therefore, novel as well. The method is desirably low cost relative to the high price of compositions currently used in the preparation of telechelic polyisobutylenes.

Although the present invention has been described in considerable detail with reference to certain embodiments, other embodiments are possible. Therefore, the spirit and scope of the appended claims should not be limited to the description of the embodiments contained herein.

1. A method for the synthesis of at least one of 1,3-di(2-methoxy-2-propyl)-5-isopropyl benzene and 1,3,5-tri(2-methoxy-2-propyl)benzene, each having high purity, comprising:
peroxidizing 1,3,5-triisopropylbenzene by gaseous oxygen in the presence of a catalyst in a basic solution to obtain a mixture of peroxide intermediates having one, two or three carboxyl groups;
reducing the mixture of peroxide intermediates and unreacted 1,3,5-triisopropylbenzene to provide a mixture of hydroxyl derivatives having one, two or three hydroxyl groups, any unreacted peroxide intermediates and any unreacted 1,3,5-triisopropylbenzene, wherein the hydroxyl derivative having two hydroxyl groups is 1,3,-di(2-hydroxyl-2-propyl)-5-isopropylbenzene, and wherein the hydroxyl derivative having three hydroxyl groups is 1,3,5-tri(2-hydroxyl-2-propyl)benzene;
separating by solvent precipitation the 1,3-di(2-hydroxyl-2-propyl)-5-isopropylbenzene and the 1,3,5-tri(2-hydroxyl-2-propyl)benzene formed in the step of reducing from any other derivatives, intermediates and unreacted materials in the reduced mixture to form a mixture of 1,3-di(2-hydroxyl-2-propyl)-5-isopropylbenzene and 1,3,5-tri(2-hydroxyl-2-propyl)benzene;
methylating a mixture of 1,3-di(2-hydroxyl-2-propyl)-5-isopropylbenzene and 1,3,5-tri(2-hydroxyl-2-propyl)benzene mixture to form a mixture of 1,3-di(2-methoxy-2-propyl)-5-isopropylbenzene and 1,3,5-tri(2-methoxy-2-propyl)benzene;
separating the 1,3-di(2-methoxy-2-propyl)-5-isopropylbenzene from the 1,3,5-tri(2-methoxy-2-propyl)benzene; and
purifying the separated 1,3-di(2-methoxy-2-propyl)-5-isopropylbenzene and the 1,3,5-tri(2-methoxy-2-propyl)benzene by crystallization from an organic solvent.

2. The method according to claim 1, wherein said catalyst in a basic solution is a Cobalt(II) salt and a tertiary amine in water.

3. The method according to claim 1, wherein the step of reducing includes reducing the mixture with sodium sulfite.

4. The method according to claim 1, wherein the step of separating by solvent precipitation uses hexane as the solvent.

5. The method according to claim 1, wherein the step of separating the 1,3-di(2-methoxy-2-propyl)-5-isopropylbenzene from the 1,3,5-tri(2-methoxy-2-propyl)benzene is carried out by vacuum distillation at different boiling points for each material.

6. The method according to claim 1, wherein the step of purifying is carried out using hexane as the organic solvent.

7. The method according to claim 1, wherein the resultant 1,3-di(2-methoxy-2-propyl)-5-iso-propylbenzene and 1,3,5-tri(2-methoxy-2-propyl)benzene is at least 95% pure.

8. The method according to claim 1, wherein the resultant 1,3-di(2-methoxy-2-propyl)-5-iso-propylbenzene and 1,3,5-tri(2-methoxy-2-propyl)benzene is at least 99% pure.

9. A composition of 1,3-di(2-methoxy-2-propyl)-5-isopropylbenzene.

10. A polymerization initiator selected from the group consisting of 1,3-di(2-methoxy-2-propyl)-5-iso-propylbenzene and 1,3,5-tri(2-methoxy-2-propyl)benzene, synthesized by the method of claim 1, and having a purity of at least 95%.

11. The polymerization initiator according to claim 10 having a purity of at least 99%.
